(19) United States
(12) Patent Application Publication
(10) Pub. No.: US 2009/0203041 A1
(43) Pub. Date: Aug. 13, 2009

(54) BMP4 INHIBITORS

(76) Inventors: Wei Shi, Cerritos, CA (US); David Warburton, La Canada, CA (US)

Correspondence Address: MICHAEL BEST & FRIEDRICH LLP, ONE SOUTH PINCKNEY STREET, P O BOX 1806, MADISON, WI, 53701, US

(21) Appl. No.: 12/297,898

(22) PCT Filed: Apr. 23, 2007
(86) PCT No.: PCT/US07/67221

§ 371 Date: Dec. 30, 2008

Related U.S. Application Data
(60) Provisional application No. 60/793,794, filed on Apr. 21, 2006.

(51) Int. Cl. G01N 33/53; C07K 7/08; C07K 14/435; C07H 21/00; C12N 5/00; C12Q 1/02; C12N 15/63; C07K 16/00

(52) U.S. Cl. 435/7.2; 530/326; 530/325; 530/324; 536/23.5; 435/325; 435/29; 435/455; 530/387.9; 436/501

(57) ABSTRACT

A fragment of Gremlin having the sequence of amino acids shown in SEQ ID NO:2 or a functional subsequence of the fragment is described. The fragment is capable of altering BMP4 activity. Also provided is a polynucleotide encoding the fragment. Also disclosed is an assay for testing a molecule for Gremlin inhibitory activity. The invention also provides a method of inhibiting BMP4 activity.

### CROSS-REFERENCE TO RELATED APPLICATIONS

This application claims priority to U.S. Provisional Application No. 60/793,794, filed Apr. 21, 2006.

### STATEMENT REGARDING FEDERALLY SPONSORED RESEARCH

This invention was supported in part by National Institutes of Health grant R01 HL068597-06. The United States government has certain rights in this invention.

### INTRODUCTION

Bone Morphogenic Proteins (BMPs) have been implicated in a variety of functions, including formation of cartilage and bone, as well as non-osteogenic development processes, e.g., organogenesis of the kidney and lung. In addition, BMPs have been found to regulate cell proliferation, migration, differentiation, and apoptosis in a number of tissues and organs. BMPs play an intrinsic role in development, as mouse embryos deficient in BMP2 or BMP4 are nonviable and die at a gestational age of 7.5 to 10.5 days.

BMP4 is a well-studied member of the BMP family. BMP4 is a potent growth factor known to play a significant role in mesodermal differentiation, basic body plan formation, and determination of the proximal-distal, left-right, and dorsal-ventral axes. BMP4 elicits different biological responses depending on the concentration of the secreted form. For example, high levels of BMP4 in early embryonic development are associated with commitment to a ventral fate, while low levels are associated with a commitment to dorsal neural and muscular tissue. Secreted active BMP4 can be inhibited at the extracellular level by interacting with secreted BMP4 antagonists, such as noggin, chordin, Cer1, DAN, and Gremlin.

BMP4 is synthesized as an inactive 50-kDa precursor protein, which dimerizes within cells via an intermolecular disulfide bond. The inactive BMP4 precursor is cleaved by members of the subtilisin-like proprotein convertase family into an active carboxyl-terminal mature BMP4 protein dimer (25 kDa for the monomer). The active mature form is secreted. The activity of BMP4 is thought to be precisely regulated extracellularly by BMP antagonists, which have a cysteine-knot structure similar to the BMPs themselves. One subset of BMP antagonists, the DAN family proteins, have a conserved eight-cysteine membered knot structure on their carboxyl termini, which distinguishes them from other BMP antagonists having a nine or 10-membered cysteine ring. The DAN family includes Gremlin, PRDC, coco, Cer1, DAN, USAG-1 and sclerostin, all of which are able to directly interact with BMPs and reduce or prevent BMP binding to its receptor. (BMPs signal via binding serine/threonine kinase receptors, both type I and type II subtypes).

Gremlin was originally identified as a molecule capable of inducing secondary axis formation in the Xenopus embryo. Gremlin has been found to associate with BMP-2, 4, and 7 in vitro and to inhibit BMP binding to cell surface receptors and BMP activities. Gremlin plays an important role in regulating proximal-distal patterning of the lung by inhibiting BMP4 signaling. Gremlin may also be involved in pathological fibrosis of the lung, kidney, and liver, such as idiopathic pulmonary fibrosis and glomerulosclerosis, through regulating other BMPs. Increased expression of Gremlin has recently been demonstrated in several models of diabetic nephropathy. In addition, Gremlin has exhibited BMP4 independent functions. For example, Gremlin has proangiogenic activity through direct interaction with endothelial cells, but not BMP4.

Nondividing and terminally differentiated cells such as neurons (or neuronal cells), alveolar epithelial cells, and goblet cells express high levels of Gremlin. The phenotypes of mice with Gremlin null mutations have shown that Gremlin plays an essential role in limb, lung and kidney development, possibly due to inappropriate BMP signaling during organ development. However, the nature of the molecular interaction between Gremlin and BMP4 was not previously well characterized.

### SUMMARY OF THE INVENTION

The inventors have demonstrated that Gremlin can specifically bind to BMP4 precursor protein inside cells, thereby preventing the production and/or secretion of mature BMP4 protein. This previously unknown mode of BMP4 inhibition indicates that Gremlin functions as a highly efficient intracellular BMP4 antagonist. The inventors have also mapped the protein sequences in Gremlin that mediate the BMP4-Gremlin interaction.

Thus, in one aspect, the invention provides a polypeptide that is a fragment of Gremlin having at least 90% identity to SEQ ID NO:2, or a functional subsequence having at least 90% identity to SEQ ID NO:2. The polypeptide is capable of altering BMP4 activity.

In another aspect, the invention provides an isolated polynucleotide which encodes a fragment of Gremlin. The fragment of Gremlin includes a sequence having at least 90% amino acid identity to SEQ ID NO:2, or a functional subsequence having at least 90% identity to SEQ ID NO:2, wherein the fragment is capable of altering BMP4 activity.

In still another aspect, the invention provides an assay for testing a molecule for Gremlin inhibitory activity. The assay entails contacting a cell with the molecule to be tested, wherein the cell expresses Gremlin, a fragment of Gremlin having at least 90% identity to SEQ ID NO:2, or a fragment of Gremlin comprising a functional subsequence having at least 90% amino acid identity of SEQ ID NO:2. In a further step, the ability of the molecule to alter secretion of mature BMP4 by the cell into the cell supernatant or an increase in BMP4 activity is assayed. An increase in secretion of BMP4 by the cell or an increase in BMP4 activity relative to a control identifies the molecule being tested as having Gremlin inhibitory activity.

In yet another aspect, the invention provides a method of altering BMP activity within a cell. The steps of the method include delivering a construct to the cell and incubating the cell under conditions that allow expression of the polynucleotide and correlating the expression of the polynucleotide with BMP4 activity, where the construct contains the polynucleotide comprising the sequence encoding a fragment of Gremlin.

In a further aspect, the invention provides an antibody capable of binding an epitope of a fragment of Gremlin. The fragment of Gremlin has at least 90% amino acid identity to SEQ ID NO:2.

In yet another aspect, the invention provides an assay for testing a molecule for Gremlin inhibitory activity. The assay entails combining the molecule, BMP4 and a labeled fragment of Gremlin comprising an amino acid sequence having at least 90% amino acid identity to SEQ ID NO:2 and assaying for binding of the labeled fragment to BMP4. A decrease in the binding of the labeled fragment to BMP4 identifies the molecule as having Gremlin inhibitory activity.

### BRIEF DESCRIPTION OF THE FIGURES

FIG. 1A is a photomicrograph depicting colocalization of BMP-4 and Gremlin proteins in mouse fetal lung tissue as visualized by cofocal microscopy. FIG. 1B is an image of an immunoblot demonstrating the communoprecipitation of BMP4 using anti-Gremlin antibodies in mouse lung and kidney tissue.

FIG. 2A is a photomicrograph depicting subcellular localization of coexpressed BMP4 tagged with Myc (Myc-BMP4) and Gremlin tagged with HA (GRE-HA) proteins by confocal microscopy. FIG. 2B is a photomicrograph depicting colocalization of Myc-BMP4 and a trans golgi specific protein, TGN38. FIG. 2C is a photomicrograph depicting colocalization of GRE-HA with TGN38. FIG. 2D is an image of an immunoblot showing the localization of Myc-BMP4 and GRE-HA into the membrane/organelle (ME) fraction of cell lysates and not the cytosol (Cy) or nucleus (Nu).

FIG. 3 is an image of an immunoblot demonstrating the intracellular interaction between expressed Myc-BMP4 and GRE-HA.

FIG. 4 depicts the inhibition of BMP-4 activity in conditioned media (CM) of COS-1 cells transfected with either Myc-BMP4 or GRE-HA or both.

FIG. 5A is an image of an immunoblot depicting the coimmunoprecipitation of Myc-BMP4 form cells expressing HA tagged Gremlin (GRE-HA) or a chimeric HA tagged Mucin-2/Gremlin (GRE-Muc-HA) and Myc-BMP4. FIG. 5B is graph depicting the BMP4 activity as measured by alkaline phosphatase of COS-1 cells expressing Gremlin (GRE-HA) or a chimeric Mucin-2/Gremlin HA tagged protein (GRE-Muc-HA) and Myc-BMP4. FIG. 5C is a graph showing the level of Myc-BMP4 mRNA levels in all cotransfected cells.

FIG. 6A is a schematic of truncated HA-tagged Gremlin molecules where the N-terminal fragment (NF) or the DAN domain (DAN) is deleted. FIG. 6B is an image of an immunoblot depicting the ability of the truncated Gremlin molecules expressed in COS-1 cells to bind co-expressed Myc-BMP4 in cell lysates and CM.

FIG. 7A is a sequence alignment between the DAN domain of Gremlin and Mucin-2, where identical sequences are highlighted in bold. FIG. 7B is a schematic diagram of the HA-tagged Gremlin/Mucin-2 chimeric proteins.

FIG. 8A is an image of an immunoblot of the coimmunoprecipitation of Myc-BMP4 with the various Gremlin/Mucin-2 chimeric proteins. FIG. 8B is a graph demonstrating the BMP4 activity of cells expressing Myc-BMP4 and the chimeric Gremlin/Mucin-2 proteins. FIG. 8C is a graph depicting the BMP4 activity of cells treated with BMP4 and the 30 amino acid fragment of Gremlin (SEQ ID NO:2).

FIG. 9A is a diagram of the ribbon structure of the Gremlin DAN domain. FIG. 9B and FIG. 9C are a diagram of the filled structure of the Gremlin DAN domain protein from the side and top, respectively.

FIG. 10 is an image of an immunoblot demonstrating the coimmunoprecipitation of Myc-BMP4 with various GRE-HA proteins with point mutations.

### DETAILED DESCRIPTION OF SEVERAL EMBODIMENTS

The inventors have found that inhibition of BMP4 activity can be achieved either by intracellular interaction of BMP4 with Gremlin when BMP4 and Gremlin are coexpressed in the same cell, or by an extracellular antagonistic mechanism if Gremlin is expressed in different cells or provided exogenously. The intracellular interaction between Gremlin and the BMP precursor protein inhibits the secretion of mature active BMP4. This efficient regulatory mechanism may direct different cell fates by regulating autocrine/paracrine activity in neighboring cells within a small microenvironment or may provide a negative feedback mechanism for certain cells. It is hypothesized that the intracellular interaction between Gremlin and BMP4 is tissue specific.

The intracellular interaction between BMP4 and Gremlin has been mapped to a 30 amino acid fragment of Gremlin that can specifically bind to BMP4 precursor protein intracellularly. This 30 amino acid fragment prevents secretion of mature BMP4 protein and efficiently down-regulates BMP4 ligand signaling. Thus, the inventors have demonstrated that Gremlin functions as an intracellular antagonist in addition to its classical extracellular antagonistic effect.

In one embodiment, the invention provides an isolated polypeptide comprising a fragment of Gremlin capable of altering BMP4 activity. A “polypeptide” is any sequence of amino acid residues joined by peptide bonds. A polypeptide may be produced artificially, e.g., by chemical synthesis, or in vitro or in vivo in a cell as described below. The polypeptide may be produced and purified from cells. Cells capable of producing the polypeptide are well known in the art, i.e. prokaryotic cells, insect cells or mammalian cells. Suitable methods for expressing the polypeptide in a cell include, e.g., transient transfection, stable transfection, or use of viral vectors for mammalian cells; baculovirus delivery for insect cells; or induced expression in bacterial cells. Methods for purifying polypeptides from a cell are well known in the art, e.g., HPLC (high pressure liquid chromatography), size exclusion chromatography, and affinity chromatography. The polypeptide may be tagged in any manner that does not affect function, e.g., a HIS or HA tag, as well known in the art.

A “fragment of Gremlin” as used herein, comprises a polypeptide having at least 90% amino acid sequence identity to SEQ ID NO:2 or having at least 90% amino acid sequence identity to a functional subsequence of SEQ ID NO:2. The sequence of SEQ ID NO:2 is conserved between mice and humans. It is to be understood that the fragment of Gremlin may include other amino acids found in the native full length Gremlin (SEQ ID NO:4) as long as it comprises at least the polypeptide defined above. In particular, the fragment is suitably at least 15 amino acids, more suitably at least, 20, 25, 30, 35, 40, 45, 50, 55, 60, 70, 80, 90 100, 125, 150, 175 or 180 amino acids in length. In some embodiments, the fragment of Gremlin may contain a sequence having at least 90% amino acid identity to SEQ ID NO:2, more suitably, at least 93%, more suitably at least 94%, 95%, 96%, 97%, 98%, 99%, or 100% amino acid identity to SEQ ID NO:2. The fragment of Gremlin may also suitably have 90% to 97% identity to SEQ ID NO:2, but not 100% identity. Percent identity may be determined using the algorithm of Karlin and Altschul (Proc. Natl. Acad. Sci. 87: 2264-68 (1990), modified Proc. Natl. Acad. Sci. 90: 5873-77 (1993)). Such algorithm is incorporated into the BLASTx program, which may be used to obtain amino acid sequences homologous to a reference polypeptide, as is known in the art.

In some embodiments, the fragment of Gremlin is a functional subsequence of SEQ ID NO:2. A “functional subsequence” is a polypeptide having 15-29 contiguous amino acids corresponding to a sequence having at least 90% amino acid identity to SEQ ID NO:2 and retaining the capability to alter BMP4 activity as described herein. However, it is to be understood that the functional subsequence may or may not alter BMP4 activity to the same degree as the sequence of SEQ ID NO:2. The subsequence may include at least 10 contiguous amino acids, more suitably 15 contiguous amino acids, more suitably 20 contiguous amino acids, more suitably 25 contiguous amino acids, more suitably 27 contiguous amino acids, and more suitably 29 contiguous amino acids of SEQ ID NO:2. The subsequence may have at least 90% sequence identity to the corresponding sequence of SEQ ID NO:2, more suitably 93%, 95%, 97%, 98%, 99% or 100%. The functional activity of the fragment can be assayed by any means known in the art for monitoring BMP4 activity as described below and in the examples, i.e., the BMP 4-stimulated C2C12 cell differentiation assay.

Although the invention is not limited to action by any particular mechanism, “altering BMP4 activity” is defined herein as an observable change, either an increase or decrease, in downstream effects of BMP4 binding to its receptor in a cell relative to a suitable control. A suitable control may be any cell which does not express the fragment. BMP activity may be monitored by detection of upregulation or downregulation of downstream signaling molecules, e.g., cytokines or receptors. For instance, as described in the examples, BMP4 activity can be measured in C2C12 cells treated with BMP4 by measuring alkaline phosphatase activity, where a decrease in phosphotase activity is a result of Gremlin inhibiting BMP4 activity.

In another embodiment, the invention provides an isolated polynucleotide comprising a sequence encoding a fragment of Gremlin capable of altering BMP4 activity. An “isolated polynucleotide” is a sequence of nucleotides which is not identical to any naturally occurring sequence of nucleotides. The term is inclusive of, for example: (a) a polynucleotide which includes a coding sequence of a portion of a naturally occurring genomic DNA molecule that is not flanked by coding sequences that flank that portion of the DNA in the genome of the organism in which it naturally occurs; (b) a polynucleotide incorporated into a vector or into the genomic DNA of a prokaryote or eukaryote in a manner such that the resulting molecule is not identical to any naturally occurring vector or genomic DNA; and (c) a cDNA molecule, a genomic fragment, a fragment produced by polymerase chain reaction, or a restriction fragment. The nucleotide sequence of SEQ ID NO:1 is one suitable sequence encoding a Gremlin fragment of the invention.

In another embodiment, the invention provides a construct comprising the polynucleotide sequence encoding the fragment of Gremlin operably linked to a promoter. A “construct” is an artificially constructed segment of nucleic acid sequence that can be introduced into cells of a target tissue or a cell in culture by way of any suitable means, e.g., a vector, including but not limited to, plasmids, cosmids, and viruses. Suitably, the promoter used in the construct includes an expression control sequence near the start site of transcription. A promoter may optionally include distal enhancer or repressor elements which may be non-contiguous with the start site of transcription. The promoter may be a “heterologous” promoter, i.e., a promoter not natively associated with the coding sequence. The promoter may be constitutive or inducible. A “constitutive” promoter is a promoter that is active under most environmental and developmental conditions. An “inducible” promoter is a promoter that is active under environmental or developmental regulation.

In another embodiment, the invention provides a cell comprising the construct described above. The cell is not limited to any particular cell type, but must be capable of expressing the polypeptide encoded by the construct under suitable conditions. Suitable cell types include eukaryotic cells, such as but not limited to animal or human cells, i.e. tumor cells, immortalized cells, primary cells, stem cells, BALB/C cells, etc. Particularly suitable cells include cells used in the examples below, i.e. COS-1 cells, C2C12 cells, and mouse lung cells. Delivery of the construct to a cell may be in vivo or in vitro. These approaches are routinely practiced in the art. For example, one of skill in the art can select any method by which a polynucleotide (e.g., DNA) can be introduced into an organelle, a cell, a tissue or an organism. Cells may be selected to study the effects of BMP4 activity on specific cell types, or may be selected as a model for diseases that are correlated with altered BMP4 or Gremlin activity. Cells used in the assay described in the Examples are also suitable. Suitable methods of administering the construct to a cell may include, but are not limited to, use of non-viral and viral vectors. Suitable viral vectors may include, but are not limited to, retroviruses (including lentiviruses), adenoviruses, adeno-associated viruses and herpes simplex virus type 1 or type 2. In vitro delivery methods include, but are not limited to, transfection, including microinjection, electroporation, calcium phosphate precipitation, using DEAE-dextran followed by polyethylene glycol, direct sonic loading, liposome-mediated transfection and receptor-mediated transfection, microprojectile bombardment, agitation with silicon carbide fibers, desiccation/inhibition-mediated DNA uptake, transduction by viral vector, and/or any combination of such methods.

The invention also provides an assay to screen for inhibitors of Gremlin. The assay involves contacting a cell expressing Gremlin or a fragment of Gremlin described herein with a molecule i.e., the putative inhibitor, and evaluating BMP4 activity or secretion of BMP4 by the cell. As used herein, “contacting a cell” refers to in vitro addition of the molecule to be tested to a cell in culture or the cell culture media. A cell may also be contacted in vivo, in any suitable subject in which the effects of BMP4 activity may be monitored, i.e. a mouse model with altered Gremlin or BMP4 activity. Molecules that can be screened in the assay include but are not limited to, peptides, proteins, protein fragments, peptidomimetics, ions, small molecules, or antibodies. “BMP4 activity” is evaluated by any suitable means known in the art. Inhibition of Gremlin activity can be monitored by release of inhibition of BMP4 activity relative to a suitable control. For example, a suitable control may be cells not contacted with the molecule being tested but otherwise treated identically to the cell contacted with the test molecule. A suitable measure of BMP4 activity known in the art is, i.e. the BMP 4-stimulated C2C12 cell differentiation assay described in the Examples. The intracellular inhibitory effects of the molecule can also be demonstrated by monitoring the level of secretion of mature BMP4 into cell supernatants, by any suitable means known in the art, e.g., ELISA, immunoblot, etc.

Although this invention is not restricted to any one mechanism, the molecule tested in the assay may induce BMP4 activity by removing Gremlin's inhibitory effect in a number of different ways. For instance, the molecule may bind to the Gremlin protein or fragment, blocking the ability of the Gremlin protein or fragment to bind BMP4, and thus releasing its inhibitory effect on BMP4 activity. Alternatively, a molecule may bind to BMP4, blocking the ability of Gremlin or a fragment of Gremlin from binding BMP4. This binding in effect blocks Gremlin's ability to inhibit BMP4 activity. In this case, the molecule is an agonist to BMP4 where it binds but does stimulate a response, thereby preventing binding by Gremlin.

The invention also provides an assay for testing a molecule for Gremlin inhibitory activity. In this embodiment, the assay measures the molecule's ability to compete for binding to BMP4 with a fragment of Gremlin labeled with a marker. Suitable markers are known in the art, such as, but not limited to, radioactivity, fluorescent markers, biotin, etc. The assay entails mixing the labeled fragment of Gremlin with the molecule being tested in the presence of BMP4, and the amount of labeled fragment is assayed in comparison to a control. The amount of labeled fragment bound to BMP4 can be assayed by any suitable means known in the art, for example, chromatography, ELISA, etc.

A further embodiment of the invention provides a method of altering BMP4 activity in a cell. The method includes delivering a construct comprising the polynucleotide sequence encoding the fragment of Gremlin to a cell capable of expressing the construct. The cells are cultured under conditions that allow expression from the promoter, typically, standard culture conditions. Expression of the construct within a cell results in inhibition of BMP4 activity. The inhibition of BMP4 activity is at least 20%, more suitably 50%, more suitably 80%, more suitably 90%, more suitably 100%, more suitably 200% relative to a control (e.g., cells treated identically, except that the promoter is not induced). Inducible promoters may be used to regulate BMP4 activity, wherein induction of the promoter results in expression of the Gremlin fragment and causes BMP4 inhibition. Levels of inhibition are then suitably compared to the control.

A further embodiment of the invention provides an antibody capable of binding an epitope of a fragment of Gremlin. The antibodies may be any of the immunoglobulin subtypes IgA, IgD, IgG, or IgM. Antibodies may be produced by any means known in the art and may be, e.g., monoclonal antibodies, polyclonal antibodies, phage display antibodies, and/or human recombinant antibodies. An “epitope of a fragment of Gremlin” is a sequence of the fragment of Gremlin that is recognized by an antibody prepared against the Gremlin fragment. The epitope may be a linear epitope, i.e. short linear amino acid sequences of the Gremlin fragment, or a conformational epitope i.e. a tertiary structural feature. The epitope may be any suitable sequence of amino acids within the fragment of Gremlin and may vary in length from 5-30 amino acids. Suitably, the epitope recognized by the antibody is at least 5 amino acids, at least 10 amino acids, at least 15 amino acids, or at least 20 amino acids in length.

The following examples are provided to assist in a further understanding of the invention. The particular materials and conditions employed are intended to be further illustrative of the invention and are not limiting upon the reasonable scope of the appended claims.

### EXAMPLES

### Example 1

### Co-localization and Interaction of BMP4 and Gremlin Proteins in Mouse Fetal Lung

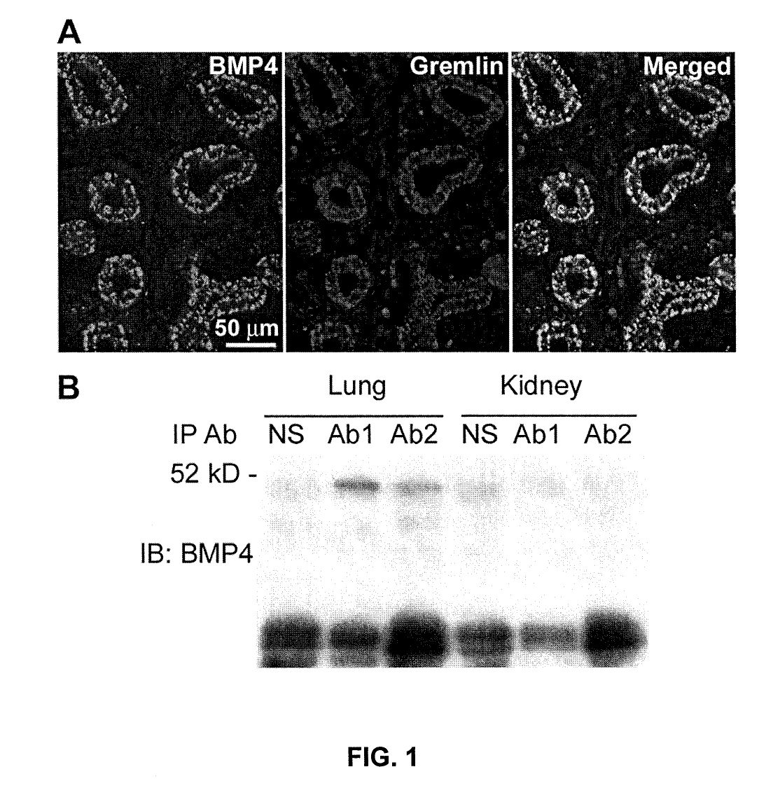

Fetal lung tissue in a mouse expresses both BMP4 and Gremlin which are thought to play an important role in lung organogenesis. To determine if there is an intracellular interaction between BMP4 and Gremlin in fetal lung cells, the endogenous protein expression patterns of these two molecules were compared by co-immunofluorescence. Co-immunofluorescence staining of endogenous BMP4 and Gremlin was performed in E14.5 embryonic mouse lung sections using rabbit anti-Gremlin and goat anti-BMP4 antibodies (Santa Cruz Biotechnology) and Alexa Fluor 594-labeled anti-goat or 488-labeled anti rabbit secondary antibodies (Invitrogen). Staining of lung sections were visualized using a Zeiss HSM510 confocal microscope with 4000× magnification and 4× digital zoom. As seen in FIG. 1A, Gremlin and BMP4 exhibit partially overlapping epithelial expression patterns.

To test whether BMP4 and Gremlin interact intracellularly, endogenous Gremlin protein was immunoprecipitated from fetal mouse lung and kidney tissue lysates by anti-Gremlin antibody. Co-immunoprecipitated BMP4 precursor protein was detected by immunoblot using anti-BMP4 antibody, and normal serum was used as a control. As seen in FIG. 1B, a 50 kDa precursor form of BMP4 was co-immunoprecipitated with Gremlin from fetal lung but not fetal kidney lysates suggesting an in vivo interaction that is cell-type specific (Ab1 from Santa Cruz Biotechnology; Ab2 generated in our laboratory).

### Example 2

### Subcellular Co-localization of Gremlin and BMP4

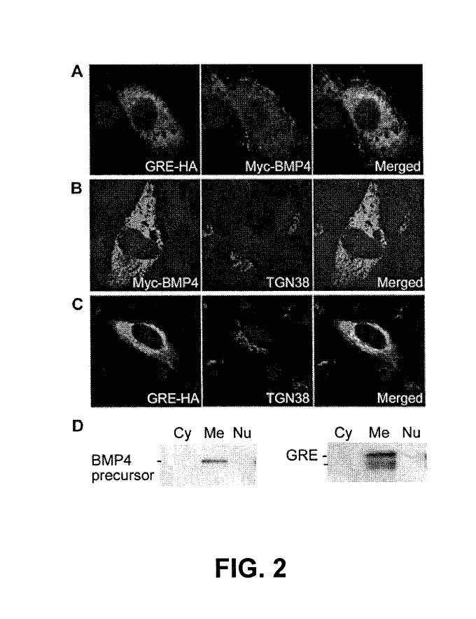

To study the intracellular interaction of BMP4 and Gremlin, cultured C2C12 cells were co-transfected with an HA-epitope tagged Gremlin (GRE-HA) and Myc-epitope tagged BMP4 (Myc-BMP4). The Myc-tagged mouse BMP4 cDNA (Myc-BMP4) was generated as described in Constam, DB and EJ Robertson, Journal of Cell Biology, 1999; 144:p. 139-149 (incorporated herein by reference), and subcloned into pcDNA3 expression vector. The Myc epitope (EQKLISEEDL) was inserted between His (296) and His (297), which is carboxyl-terminal to the cleavage site Arg (292)-Ser (293) of mature BMP4 so that both precursor and mature forms of exogenously expressed BMP4 could be detected by Myc-epitope antibodies. HA-tagged mouse Gremlin (GRE-HA) was generated in our lab as described in Shi et al, American Journal of Physiology-Lung Molecular and Cellular Physiology 2001, 280:L1030-1039. Tagged BMP4 and Gremlin were co-expressed in C2C12 cells, and co-immunofluorescence staining with c-Myc antibodies (Santa Cruz) and anti-HA antibodies (Covance) and visualized with a confocal microscope. As seen in FIG. 2A, GRE-HA and Myc-BMP4 co-localized to the cytoplasm of the cells.

For more specific subcellular localization, cells expressing GRE-HA and Myc-BMP4 were immunostained with anti-Myc, or anti-HA antibodies and anti-TGN38 (a marker for the Trans-Golgi network) antibodies (Santa Cruz) and visualized as described in Example 1. Cell nuclei were stained with 4′6-diamidino-2-phenylindole (blue color). As seen in FIGS. 2B and 2C, both BMP4 and Gremlin co-localized with TGN38 suggesting they trafficked through similar intracellular compartments.

The subcellular localizations of Myc-BMP4 and GRE-HA was confirmed by Western blot detection in different subcellular fractions. As seen in FIG. 2D, both the BMP4 precursor and Gremlin were detected in the membrane/organelle protein extract fraction of cell lysates (including endoplasmic reticulum and Golgi) but not in the cytosolic or nucleic protein extract fractions (Cy, cytosol; Me, membrane/organelle; Nu, nuclei).

### Example 3

### Intracellular Interaction Between BMP4 and Gremlin

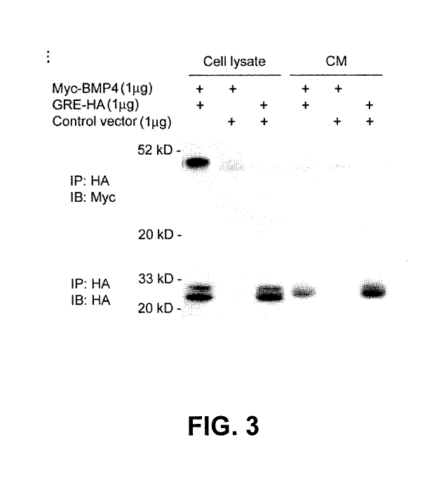

To test whether exogenous BMP4 and Gremlin physically interact intracellularly, Myc-BMP4 and GRE-HA were co-transfected into COS-1 cells using Lipofectamine Plus (Invitrogen). Transfected cells were first cultured in medium containing 10% fetal calf serum for 20 hours and then cultured in serum-free conditioned medium (CM) for an additional 48 hours, and experiments were repeated at least three times. Co-immunoprecipitation using anti-HA antibodies of the cell lysate and conditioned medium (CM) demonstrated that Myc-BMP4 precursor was found in the cell lysate, but no mature Myc-BMP4 protein was found in the conditioned medium (CM), as seen in FIG. 3 by Western blot using anti-Myc antibodies.

### Example 4

### Gremlin Expression Inhibits BMP4 Activity

To test whether exogenous expression of Gremlin can inhibit the activity of exogenously expressed BMP4, the activity of BMP4 secreted into the CM was measured by its effect on mouse myoblast C2C12 cell differentiation into osteoblasts. C2C12 cells were grown in 96-well plate until 90% confluence. Agents (100 ng/ml BMP4 or equivalent amounts of concentrated conditioned medium) were then added into the culture medium (Dulbecco's modified Eagle's medium with 5% fetal bovine serum) and the cells were cultured for 3 days. The cells were then lysed with 0.1% Triton X-100, and the alkaline phosphatase (ALP) activity in the cell lysate was quantified by adding substrate p-nitrophenyl phosphate and measuring absorbance at A₄₀₅. Triplicate measurements were preformed for every sample. Conditioned media from cells transfected with control, Myc-BMP4, GRE-HA, or Myc-BMP4 and GRE-HA were assayed for BMP4 activity.

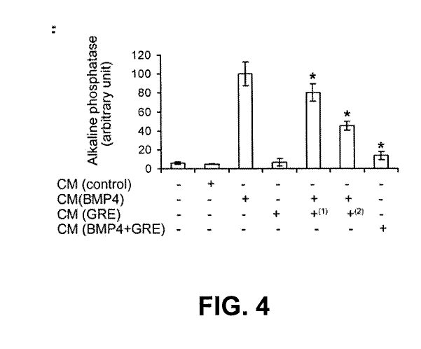

Results are reported in FIG. 4. CM collected separately from cells transfected with Myc-BMP4 or GRE-HA was mixed and preincubated for 1 hour before adding to C2C12 cells (1). COS-1 cells were transfected separately with Myc-BMP4 or GRE-HA, trypsinized and co-cultured during CM collection (2). BMP4 activity in the CM of Myc-BMP4 transfected cells was inhibited by 20±9% when an equal amount of CM collected from GRE-HA transfected cells was added, and BMP4 activity in CM collected from cells that had been transfected separately with Myc-BMP4 or GRE-HA exhibited higher BMP4 inhibitory effect, 55±5%. BMP4 activity in cells co-transfected with Myc-BMP4 and GRE-HA was markedly inhibited, 86±4% (p<0.05), suggesting a more efficient inhibitory effect of intracellular Gremlin-BMP-4 interaction than extracellular antagonistic effect.

### Example 5

### Intracellular BMP4-Gremlin Interaction Prevents Mature BMP4 Secretion

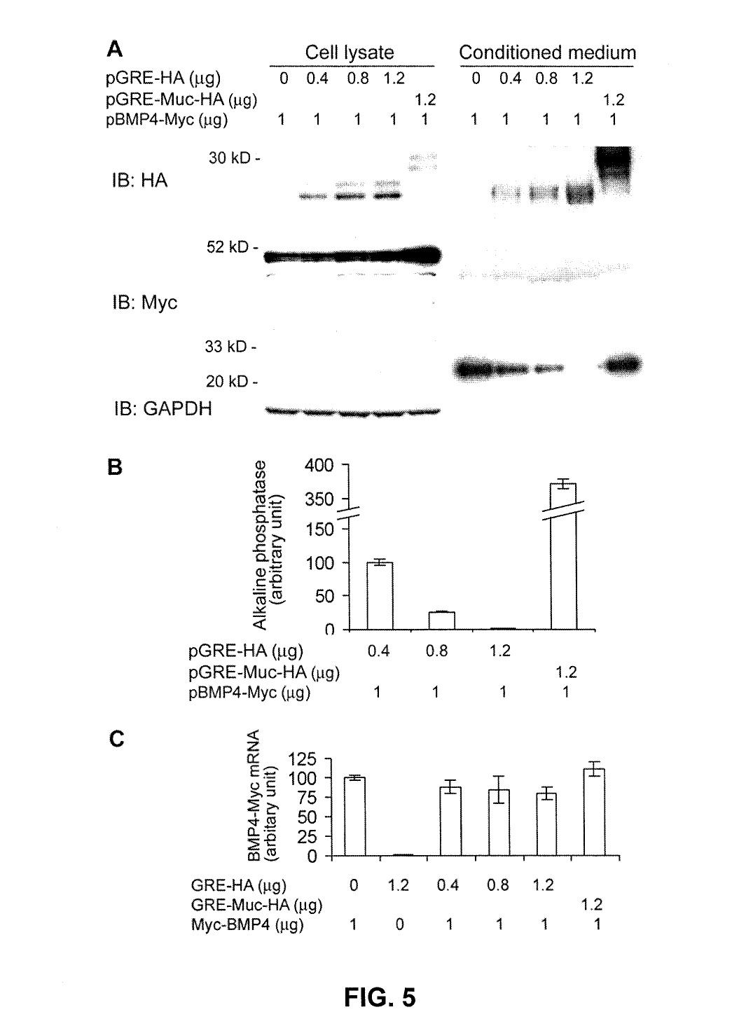

To assay cells to test if the BMP4-Gremlin interaction prevents mature BMP4 secretion from cells, COS-1 cells were co-transfected with GRE-HA, GRE-Muc-HA (a mutated Gremlin that does not bind BMP4), Myc-BMP4 or a combination of the vectors, and expression of GRE-HA and Myc-BMP4 was detected in the cell lysates and conditioned media (CM) by Western blot. GRE-Muc-HA was created similar to the chimeras described below in Example 7. Glyceraldehyde-3-phosphate dehydrogenase (GAPDH) was used as a protein loading control for cell lysates. As seen in FIG. 5A, no mature BMP4 was detected in the cell lysates, whereas mature but no precursor BMP4 was detected in the concentrated CM, and reduced amounts of secreted mature BMP4 protein detected in the CM were inversely proportional to intracellular Gremlin expression. FIG. 5A also shows that the level of BMP4 precursor protein in the cell lysate was not significantly increased even though BMP4 secretion was reduced. Both secreted forms of Gremlin and mature Myc-BMP4 in CM were increased when co-expressed with Gremlin that was mutated to lose BMP4 binding activity (GRE-Muc-HA), while the level of mutated Gremlin was low in cell lysate, as shown in FIG. 5A. Thus, intracellular interaction of Myc-BMP4 precursor and GRE-HA may prevent Myc-BMP4 precursor processing and mature Myc-BMP4 secretion.

Detection of BMP4 secretion was also measured by assaying the CM for BMP activity by measuring the effects of the CM on C2C12 cell differentiation in osteoblast cells as described in Example 4. Equivalent amounts of CM were added to the C2C12 cell culture medium. As seen in FIG. 5B, BMP4 activity inhibition was consistent with reduced levels of secreted mature Myc-BMP4 protein levels in CM detected by Western blot in FIG. 5A.

To determine if the changes in mature Myc-BMP4 levels in the CM were due to differences in BMP4 gene expression, the mRNA levels of Myc-BMP were determined by quantitative real-time PCR using Myc-specific primers and a protocol published previously in Shi et al., Developmental Biology, 2003:261, p 371-380 (incorporated herein by reference in its entirety). As shown in FIG. 5C, the expression levels of Myc-BMP4 mRNA was similar in all samples transfected and expressing Myc-BMP4.

### Example 6

### Gremlin DAN Domain is Essential to Mediate Gremlin-BMP4 Interaction

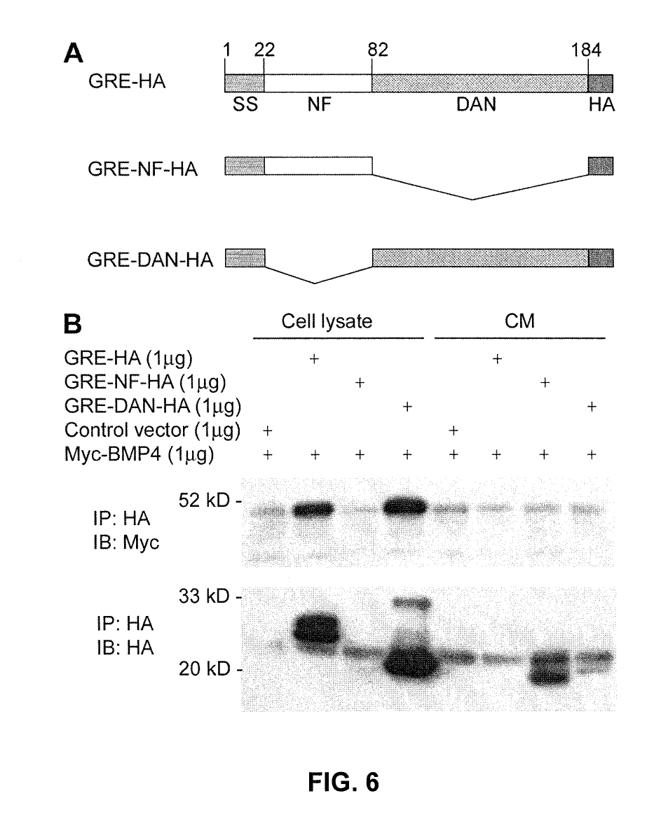

To determine the specificity of BMP4-Gremlin binding and the molecular motif of Gremlin that is responsible for the intermolecular interaction, two truncated Gremlin molecules, GRE-DAN-HA and GRE-NF-HA, were generated with truncation of the NH₂-terminal fragment (NF) or the carboxyl-terminal DAN domain (DAN), respectively, as diagramed in FIG. 6A (amino acid positions are in reference to SEQ ID NO:4). The two truncated Gremlin molecules were co-transfected with Myc-BMP4 into COS-1 cells, and immunoprecipitation was performed with anti-HA antibodies. As seen in FIG. 6B, removal of the DAN domain from Gremlin completely abolished the ability of Gremlin to bind BMP4 and greatly increased the secretion of Gremlin in the CM. Gremlin that retained its BMP4 binding activity (GRE-DAN-HA and GRE-HA) were retained in the cell lysates.

### Example 7

### Mapping of the BMP-Binding Motif of Gremlin DAN Domain Isolates a 30 Amino Acid Fragment

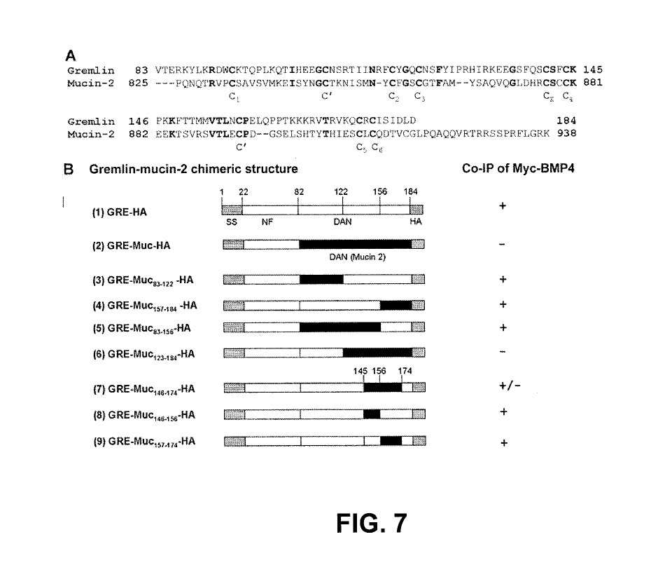

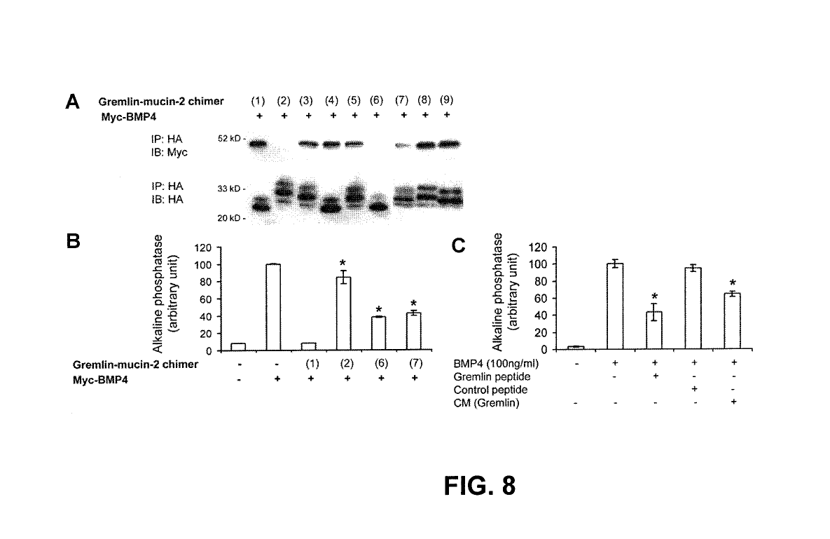

To map the BMP-4 binding motif within the Gremlin DAN domain, chimera molecules were created using the Gremlin DAN domain and Mucin-2, a protein with a highly conserved eight-member Cys-knot DAN domain homology. Mucin-2 is unable to bind BMP4. FIG. 7A shows the amino acid alignment of the DAN domain of Gremlin and the equivalent domain in Mucin-2, where the conserved residues are in bold, and the cysteines that form the Cys-knot are marked by a “C”. As shown in FIGS. 8A and 8B, replacement of the entire DAN domain of Gremlin with the domain of Mucin-2 completely abolished BMP4 binding activity and BMP4 inhibitory function.

Partial cDNA of mouse Mucin-2, which encodes a Mucin-2 protein fragment of 114 amino acids on the carboxyl terminus (PQNQ . . . LGRK, GenBank™ accession number XP_{—}620590) was generated by high fidelity RT-PCR (Pfu, Stratagene) and verified by DNA sequencing. An HA epitope was also added to the carboxyl terminus of Mucin-2 by inverted PCR. A variety of Gremlin-Mucin-2 chimeric cDNA constructs were created by inverted PCR in combination with restricted digestion and ligation of PCR products. The Gremlin-Mucin-2 chimeras generated are as diagrammed in FIG. 7B. These HA-epitope tagged Gremline-Mucin-2 chimera proteins were co-expressed with Myc-BMP in COS-1 cells by transient transfection. The BMP4 binding activities and inhibitory activities were evaluated by immunoprecipitation and BMP4-stimulated C2C12 cell differentiation assay as described in Examples 3 and 4. The results for the Gremlin-Mucin-2 chimeras are seen in FIGS. 8A and 8B. A 30 amino acid region corresponding to Gremlin amino acid sequence 145-174 of full length Gremlin (SEQ ID NO:4), PKKFTTMMVTLNCPELQPPTKKKRVTRVKQ (SEQ ID NO:2) is sufficient to mediate the interaction between Gremlin and BMP4 and Gremlin's inhibitory effect as seen in FIG. 8C.

The function of the 30 amino acid Gremlin motif in mediating interaction between Gremlin and BMP4 and BMP4 inhibitor activity was assayed by the BMP4-induced C2C12 cell differentiation assay. A linear peptide corresponding to the mapped amino acid sequence (145-174) was synthesized by Genscript (Scotch Plains, N.J.). As seen in FIG. 8C, preincubation of the linear peptide (5 μm) with BMP4 (100 ng/mL), significantly inhibited BMP4 activities by 56±10% (p<0.05). A control peptide with the amino acids sequence of Gremlin 37-52 (5 μm) did not inhibit BMP4 signaling activity.

### Example 8

### Structure Modeling of the Gremlin DAN Domain

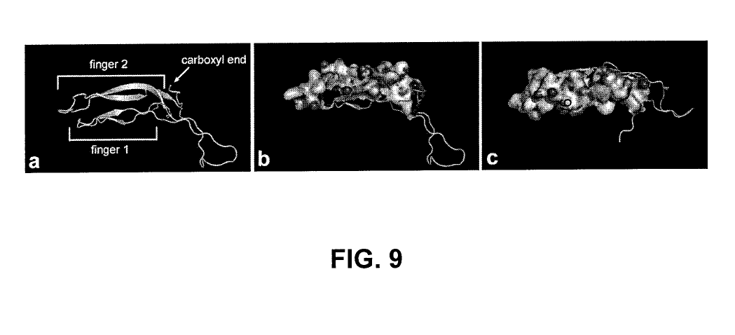

FIG. 9 is a 3D molecular model depicting the approximate structure of the mouse Gremlin DAN domain (amino acids 94-184 of SEQ ID NO:4) which was determined with respect to the published structure of human chorionic gonadotropin chain B by homology modeling using the program Modeler version 8.1. As described by Avsian-Kretchmer and Hsuch (Avsian-Kretchmer, O and Hsuch, A. J., Molecular Endocrinology, 200, 18:p. 1-12), human chorionic gonadotropin chain B provides a suitable homology modeling template for Gremlin because they both share cysteine knots that form eight-membered rings and have “fingers” of similar size. The mapped 30-amino region (145-174 of SEQ ID NO:4) spans the whole finger structure (“finger 2”), as depicted in FIG. 9A, providing a surface that is independent of the rest of the molecule. Finger 2 also contains positively charged lysine and arginine residue side chains across the surface, suggesting a positively charged surface. FIGS. 8B and 8C show the side and top interface, respectively, of the Gremlin domain in a filled structure, respectively.

### Example 9

### Partial Substitutions within the 30 Amino Acid Fragment of Gremlin does not Effect Gremlin Function

To test whether changes could be made to the 30 amino acid fragment of Gremlin without effecting its inhibition and binding to BMP-4, substitutions within the full length Gremlin domain were prepared shown in Table 1.

TABLE 1

| | |
|---|---|
| Gremlin protein | Sequence |

| | |
|---|---|
| Wildtype | PKKFTTMMVTLNCPELQPPTKKKRVTRVKQ (SEQ ID NO:2) |
| Mutant 5 | A-A--------------------------- |
| Mutant 6 | ---------AA--A----------------- |
| Mutant 7 | ----------------------A------- |

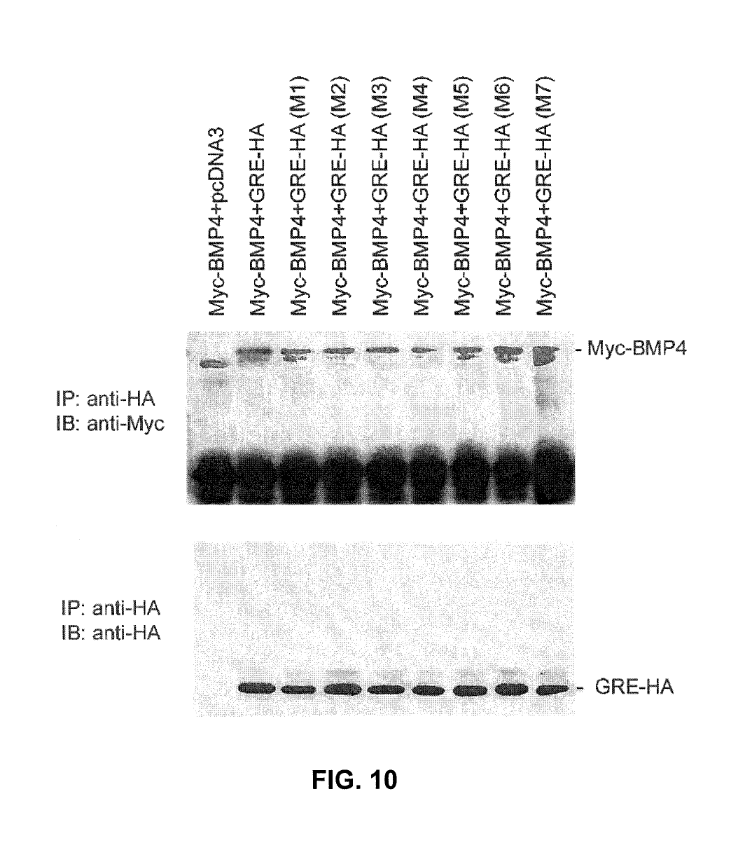

The point mutations of the Gremlin protein did not abrogate Gremlin-BMP4 interaction, as illustrated by protein co-immunoprecipitation of Myc-BMP4 and the mutant Gremlin fragments in FIG. 10.

While the compositions and methods of this invention have been described in terms of exemplary embodiments, it will be apparent to those skilled in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention. In addition, all patents and publications listed or described herein are incorporated in their entirety by reference.

As used in this specification and the appended claims, the singular forms “a,” “an,” and “the” include plural referents unless the content clearly dictates otherwise. Thus, for example, reference to a composition containing “a polynucleotide” includes a mixture of two or more polynucleotides. It should also be noted that the term “or” is generally employed in its sense including “and/or” unless the content clearly dictates otherwise. All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

It also is specifically understood that any numerical value recited herein includes all values from the lower value to the upper value, i.e., all possible combinations of numerical values between the lowest value and the highest value enumerated are to be considered to be expressly stated in this application.

1. A isolated polypeptide comprising a fragment of Gremlin, the fragment comprising:
a) an amino acid sequence of SEQ ID NO:2;
b) a functional subsequence of SEQ ID NO:2; or
c) an amino acid sequence having 90% amino acid identity to a) or b),
wherein the polypeptide is capable of altering BMP4 activity.

2. The polypeptide of claim 1, wherein the fragment comprises a sequence having 90 to 97% amino acid identity with SEQ ID NO:2.

3. An isolated polynucleotide encoding the polypeptide of claim 1.

4. The polynucleotide of claim 3 comprising the nucleotide sequence of SEQ ID NO:1.

5. The polynucleotide of claim 3, wherein the fragment comprises the amino acid sequence of SEQ ID NO 2.

6. A construct comprising the polynucleotide of claim 3 operably linked to a promoter.

7. The construct of claim 6, wherein the promoter is an inducible promoter.

8. The construct of claim 6, wherein the promoter is a constitutive promoter.

9. A cell comprising the construct of claim 6, wherein the cell expresses the polypeptide encoded by the construct.

10. An assay for testing a molecule for Gremlin inhibitory activity comprising:
a) contacting a cell with a molecule, wherein the cell expresses:
i) Gremlin; or
ii) the polypeptide of claim 1;
b) evaluating levels of BMP4 secretion by the cell or BMP4 activity,
wherein increased BMP4 secretion or increased BMP4 activity relative to a control identifies the molecule as having Gremlin inhibitory activity.

11. The assay of claim 10, wherein the molecule is a peptidomimetic.

12. The assay of claim 10, wherein the molecule having Gremlin inhibitory activity binds BMP4.

13. The assay of claim 10, wherein the molecule binds to Gremlin or the fragment of Gremlin.

14. A method of altering BMP4 activity comprising delivering the construct of claim 6 to a cell and incubating the cell under conditions that allow expression of the polynucleotide, wherein expression is correlated with altered BMP4 activity.

15. The method of claim 13, wherein altering BMP4 activity comprises inhibiting BMP4 activity.

16. The method of claim 14, wherein BMP4 activity is inhibited at least 50% compared to a control.

17. An antibody capable of binding an epitope of a fragment of Gremlin having at least 90% amino acid identity to SEQ ID NO:2.

18. The antibody of claim 17, wherein the fragment comprises a sequence having 90 to 97% amino acid identity with SEQ ID NO:2.

19. The antibody of claim 17, wherein the fragment comprises the amino acid sequence of SEQ ID NO:2.

20. An assay for testing a molecule for Gremlin inhibitory activity comprising:
a) combining the molecule, BMP4 and a labeled fragment of Gremlin comprising an amino acid sequence having at least 90% amino acid identity to SEQ ID NO:2; and
b) assaying for binding of the labeled fragment to BMP4,
wherein a decrease in the binding of the labeled fragment to BMP4 identifies the molecule as having Gremlin inhibitory activity.


SEQUENCE LISTING (7 sequences)

SEQ ID NO: 1 - DNA, 90 bp, Homo sapiens
cccaagaagttcaccaccatgatggtcacactcaactgtcctgagctacagccacccaccaagaagaaaagggtcacacgcgtgaagcag

SEQ ID NO: 2 - protein, 30 aa, Homo sapiens
PKKFTTMMVTLNCPELQPPTKKKRVTRVKQ

SEQ ID NO: 3 - DNA, 552 bp, Mus musculus
atgaatcgcaccgcatacactgtgggagcgttgcttctcctcctggggaccctactgccaacagctgaggggaaaaagaaaggttcccaaggagccattccgcctcctgacaaggctcagcacaatgactctgagcagacccagtccccaccacaacctggctccaggacccgggggcggggccaggggcggggcaccgccatgcctggagaggaggtgcttgagtccagccaagaggccctgcacgtgacagagcgcaagtatctgaagcgagattggtgcaaaactcagcccctgaagcagaccatccacgaggagggctgcaacagccgcactatcatcaaccgcttctgttatggccagtgcaactccttctacatccccaggcacatccgaaaggaggaagggtcctttcagtcttgctccttctgcaagcccaagaagttcaccaccatgatggtcacactcaactgtcctgagctacagccacccaccaagaagaaaagggtcacacgcgtgaagcagtgccgttgcatatccatcgacttggat

SEQ ID NO: 4 - protein, 184 aa, Mus musculus
MNRTAYTVGALLLLLGTLLPTAEGKKKGSQGAIPPPDKAQHNDSEQTQSPPQPGSRTRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 5 - DNA, 552 bp, Homo sapiens
atgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggat

SEQ ID NO: 6 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

SEQ ID NO: 7 - protein, 114 aa, Mus musculus
PQNQTRVPCSAVSVMKEISYNGCTKNISMNYCFGSCGTFAMYSAQVQGLDHRCSCCKEEKTSVRSVTLECPDGSELSHTYTHIESCLCQDTVCGLPQAQQVRTRRSSPRFLGRK